(19) United States
(12) Patent Application Publication
(10) Pub. No.: US 2025/0334592 A1
(43) Pub. Date: Oct. 30, 2025

(54) CU GROWTH ENHANCED PLASMONIC ASSAY FOR ISOLATION-FREE EXOSOME ANALYSIS

(71) Applicant: The Administrators of the Tulane Educational Fund, New Orleans, LA (US)

(72) Inventors: Tony Hu, New Orleans, LA (US); Shu Wang, New Orleans, LA (US)

(21) Appl. No.: 18/866,141

(22) PCT Filed: May 16, 2023
(86) PCT No.: PCT/US2023/022420

§ 371 Date: Nov. 15, 2024

Related U.S. Application Data
(60) Provisional application No. 63/343,067, filed on May 17, 2022.

(51) Int. Cl. G01N 33/68; G01N 33/553; G01N 33/569; G01N 33/574

(52) U.S. Cl. CPC G01N 33/6893; G01N 33/553; G01N 33/5695; G01N 33/57492; G01N 2469/10

(57) ABSTRACT

An assay for detection of a target extracellular vesicle in a sample advantageously utilizes copper shells grown on gold nanostructures labeling the target extracellular vesicle to enhance the scattering signal and thereby improve assay sensitivity.

### BACKGROUND

Extracellular vesicles are secreted from different types of cells and can ultimately accumulate and circulate in a variety of body fluids. Due to the unique role of extracellular vesicles in biogenesis processes, extracellular vesicles secreted from diseased cells can preserve important biomolecules including proteins and nucleic acids. These biomolecules are closely associated with the progression and prognosis of many diseases, such as various cancers, hepatic disease, and infectious diseases. As such, extracellular vesicles may be able to serve as new markers for disease diagnosis and prognosis. Although, extracellular vesicles are generally secreted and abundantly present in circulation, extracellular vesicles from diseased cells constitute only a small portion of extracellular vesicles present in body fluid, particularly in early stages of a disease. Conventional methods required time-consuming, labor-intensive procedures, requiring isolation steps and subsequent immune/molecular approaches to detect molecular contents of interest carried by the extracellular vesicles.

Conventional technologies utilize plasmonic nanosensors. For example, enhanced Rayleigh scattering from gold nanospheres/gold nanorods) has enabled isolation-free, nanoplasmonic enhanced scattering (nPES) methods for extracellular vesicle detections. Antibody-conjugated gold nanoparticles have also been used to label target extracellular vesicles to induce strong scattering signals for imaging and quantification. However, there remains a trade-off between sensitivity and throughput, as well as instrument accessibility. For example, high-magnification analyses can enable ultrasensitive detection, but requires skilled users and expensive optical settings that are not easily accessible. In contrast, low-magnification analysis can provide a cost-effective, even portable approach, but the sensitivity is not sufficient to detect low concentrations of target extracellular vesicles.

Several strategies have also been developed to modulate the plasmonic signals of gold nanoparticles, including assembly of multiple gold nanoparticles and etching. However, aggregation/disaggregation of nanoparticles requires specifically designed crosslinkers, additional washing steps, and can suffer from low efficiency on solid substrates. Surface etching of gold nanoparticles through dissolution of Au⁰ by oxidation to Au¹ requires a strong oxidizing agent that could potentially disrupt the extracellular vesicles and adversely affect the results. Also, smaller gold nanoparticles are generally considered to show a lower Rayleigh scattering that could weaken the signal.

### SUMMARY

A need is present for an improved assay for sensing trace amounts of target extracellular vesicles. The assays and methods of the disclosure enhance the signal for nanoplasmonics to enhance scattering signals for extracellular vesicle detection. Assays and methods of the disclosure can have increased sensitivity and provide a one-step process. The processes of the disclosure can be performed in shorter timeframes, such as about 10 min, making them more clinically applicable.

In accordance with the disclosure, an assay for detecting target extracellular vesicles in a sample comprising or suspected to be comprising target and non-target extracellular vesicles can include providing a substrate having a capture antibody coated thereon, the capture antibody capable of binding with the target and non-target extracellular vesicles; contacting the substrate with the sample under conditions sufficient to allow for binding of the target and non-target extracellular vesicles with the capture antibody, thereby immobilizing the target and non-target extracellular vesicles on the substrate; contacting the substrate having the immobilized target and non-target extracellular vesicles with a labeling reagent comprising gold nanostructures associated with a detection antibody, the detection antibody being capable of binding only to the target extracellular vesicles, wherein upon binding of the detection antibody with the target extracellular vesicles, the target extracellular vesicles are labeled with the gold nanostructures; incubating the substrate comprising the labeled target extracellular vesicles with a copper growth reagent under conditions sufficient to induce copper ions to reduce and form a copper shell surrounding the gold nanostructure, the copper growth reagent comprising copper ions, a reductant, and a structure directing agent; and detecting a scattering signal from the gold nanostructures having the copper shell formed thereon.

In accordance with the disclosure, a kit for performing an assay for detecting target extracellular vesicles in a sample comprising or suspected to be comprising target and non-target extracellular vesicles, the kit can include a substrate coated with a capture antibody, the capture antibody capable of binding with the target and non-target extracellular vesicles; a labeling reagent comprising gold nanostructures associated with a detection antibody; a copper growth reagent, the copper growth reagent comprising copper ions, a reductant, and a structure directing agent; and instructions for performing the assay comprising contacting the substrate coated with the capture antibody with the sample to immobilize the target and non-target extracellular vesicles on the substrate; contacting the immobilized target and non-target extracellular vesicles with the labeling reagent comprising the gold nanostructure associated with the detection antibody, whereby the detection antibody selectively binds with the target extracellular vesicles to thereby label the target extracellular vesicles, and incubating the substrate comprising the labeled target extracellular vesicles in the copper growth solution to form a copper shell around the gold nanostructure, and detecting a scattering signal from the gold nanostructures having the copper shell formed thereon.

### BRIEF DESCRIPTION OF THE DRAWINGS

FIG. 1 is a schematic illustration of an assay in accordance with the disclosure.

FIG. 2A is a dark field image before Cu growth enhancement.

FIG. 2B is a dark field image after Cu growth enhancement.

FIG. 3A includes (i) SEM and (ii) TEM images of bare Au nanorods.

FIG. 3B is a graph showing the surface plasmonic reaction of the bare gold nanorods (50 nm).

FIG. 3C includes (i) SEM and (ii) TEM images of Au nanorods having copper shells grown thereon in accordance with the disclosure.

FIG. 3D is a graph showing the surface plasmonic reaction of the gold nanorods having the copper shells grown thereon (200 nm).

FIG. 4A is a representative dark field image of scattering signals from Cu enhancement on gold nanorods with a 2-fold serial dilution from 625 μg/mL to 0 μm/mL.

FIG. 4B is a representative dark field image of scattering signals from Cu enhancement on gold nanospheres with a 2-fold serial dilution from 625 μg/mL to 0 μm/mL.

FIGS. 5A and 5B are graphs showing correlation of scattering single verses initial NP concentration for (A) AuS and (B) AuR samples with and without exposure to Cu nanoshell growth conditions (dashed lines depict log-log correlations, horizontal lines indicate the signals generated from blank substrates; gray coloring columns indicate signals beyond linear range).

FIG. 6A is nanoparticle (NP) scattering intensity detected after 5 min Cu nanoshell growth on an AuR core with no reductant, with added sodium citrate, glutathione (GSH), sodium borohydride (NaBH₄), or NaVc, or NaVc without AuR.

FIG. 6B is NP scattering signal detected after 10 min Cu nanoshell growth with AuR and NaVc in the presence the indicated Cu²⁺ ion and PEI ratios.

FIG. 7 is a graph showing extracellular vesicle detection performance of ELISA, an assay using bare gold nanorods and an assay in accordance with the disclosure using copper enhanced gold nanorods; the assay in accordance with the disclosure demonstrated the widest dynamic range and the LOD reached 1800 EVs/μl.

FIG. 8A is a graph of LAM-EV signal detected in serum from pediatric TB (confirmed and unconfirmed) and non-TB (unlikely) cases as measured by an AuR assay.

FIG. 8B is a graph LAM-EV signal detected in serum from pediatric TB (confirmed and unconfirmed) and non-TB (unlikely) cases as measured by Cu-NEI.

FIG. 8C is a graph showing ROC curves and AUC values (in parentheses) for serum LAM EV AuR and Cu-NEI signal, and binary (positive/negative) sputum Xpert MTB/RIF and urinary LAM assay results.

FIG. 9A is a graph showing exoPD-L1 level detected by Cu-NEI in Cu-NEI enable the PD-L1+ analysis from NSCLC cohort.

FIG. 9B is a graph showing exoPD-L1 level detected by ELISA.

FIG. 9C is graphs showing PD-L1 biopsy score comparation from good response group and poor response group.

FIG. 9D is an ROC curve of three different methods of FIGS. 9A-9C.

FIG. 9E is a graph showing the combined response of ExoPD-L1 Cu-NEI and biopsy score;

FIG. 9F is a graph showing the combined response of ExoPD-L1 ELISA and biopsy score.

FIG. 9G is a graph showing the Pearson correlation of ExoPD-L1 and PFS.

FIG. 9H is a graph showing the Pearson correlation of biopsy and PFS.

FIGS. 10A and 10B are selected area electron diffraction (SAED) patterns of (A) an AuR and (B) AuS core.

FIGS. 10C and 10D are SEM images of (C) AuR@Cu and (D) AuS@Cu NPs (scale bar: 500 nm).

FIGS. 10E and 10F are simulated optical scattering and electric field produced by plane wave interactions with (E) tetrahedral or (F) a cubical nanoshell models and the adjacent atmosphere.

FIGS. 10F and 10G are graphs showing total heat losses for (G) Cu NP cubes and tetrahedrons with edges of 200 nm and (H) Cu NP cubes with the indicated ½ edge lengths.

FIG. 11A includes TEM images of Cu nanoshells formed on AuR and AuS NPs (Scale Bar: 100 nm).

FIG. 11B is an image showing distribution of AuR@Cu and Aus@Cu nanoshell geometries, as determined by counting the number and percentage of each shape in three SEM images for each sample (239 AuR@Cu and 188 AuS@Cu particles).

FIG. 11C is an image showing calculated scattering profiles of cubic and spherical Cu nanoshells with 200 nm edges and diameters as simulated by COMSOL, deriving the complex refractive index of copper sphere and cube from the Optical Materials Database, where the heatmap bar indicates W/m³.

FIGS. 11D and 11E include DFM images of the overall and single NP (inset) scattering signal produced by (D) AuR and AuR@Cu NPs and (E) AuS and AuS@Cu NPs.

FIG. 12A shows time lapse DFM images of a AuR@Cu nanoshell in situ growth reaction (scale bar=200 μm)

FIG. 12B shows a heatmap contour graph, corresponding to FIG. 12A, of its scattering signal intensity, indicating pixel intensity on a 0 to 255 scale.

FIG. 12C shows scattering signal during a 20 min recording of AuR and AuS samples in incubated in the presence (AuR@Cu and AuS@Cu) or absence (AuR and AuS) of Cu nanoshell growth reagents.

FIG. 12D is NP scattering intensity detected in at the indicated times in AuR@Cu and AuS@Cu growth reactions after subtraction of their baseline AuR or AuS signal (mean+SE of triplicate reactions shown in FIGS. 6A and 6B).

FIG. 13 includes DFM images of Cu nanoshell growth reactions precipitates after input AuR NPs have been incubated with Cu²⁺ solution and PEI supplemented with the indicated reducing agents.

FIG. 14A is a graph showing signal change of AuR@Cu vs AuR and Aus@Cu vs AuS NPs.

FIG. 14B is a graph showing optimization of input AuR concentration for the Cu-NEI platform.

FIGS. 14C and 14D are graphs showing normalized intensity for serial dilutions of (E) IgG standard measured by AuR immunoassay and Cu-NEI, and (F) EV standard analyzed by ELISA and Cu-NEI, where data was normalized against the blank control of its respective assay and graphed to indicate its correlation with a four-parameter logistic curve. All graphs depict mean+SE values of three technical replicates for each sample.

FIG. 15A is a TEM image (scale bar: 200 nm) of an EV standard sample isolated from the HCT116 cell line.

FIG. 15B is a graph of NTA-determined size distribution of an EV standard sample isolated from the HCT116 cell line.

FIG. 15C is an image showing Western blot data for the expression of two EV markers (CD81 and CD63) in protein lysates of these EV samples.

FIG. 15D is a graph showing the correlation between NTA-quantified EV concentration and BCA-measured EV protein concentration (R²=0.98).

FIG. 16A is a TEM image of an HCT116 cell-derived EV immunolabeled with an CD63 antibody-labeled AuR probe (scale bar: 100 nm).

FIG. 16B is a graph showing NTA-measured EV size distributions in EV samples incubated with Cu nanoshell growth reagents with (A) or without (B) BSA-blocked AuRs, or added PBS(C), and then centrifuged to remove AuR@Cu or self-nucleated CuNPs. Differences were analyzed by one-way ANOVA with Bonferroni's correction.

FIG. 16C is a graph showing EV concentrations and recovery rates detected before (gray bars) and after EV standard samples were incubated with unblocked (A) or BSA-blocked (B) AuR@Cu NPs and centrifuged to remove these NPs.

FIG. 17A is an image showing LAM expression detected with EVs isolated from Mtb-infected or non-infected THP-1 or Mtb culture filtrate protein (CFP) used as a positive control.

FIG. 17B is an image showing specificity of the LAM-EV Cu-NEI as determined using EVs derived from THP-1 macrophage cultures exposed to microbial pathogens responsible for human infections, including Streptococcus pneumonia (S.pn), Klebsiella pneumonia (K.pn), Escherichia coli (E. coli), Staphylococcus aureus (S. a) and Pseudomonas aeruginosa (Ps.a). Data indicate the mean+SE values for triplicate technical replicates and *p<0.05, **p<0.01, and ***p<0.001 differences determined by a one-way parametric ANOVA with Dunnett post-tests.

FIG. 18 Schematic of the Cu-NEI approach in which monocrystalline (vs. polycrystalline) nanoshell growth on an NP probe for a target biomarker markedly enhances assay sensitivity.

FIG. 19A is a graph showing AuR and Cu-NEI EV-LAM signal intensity for TB and Non-TB samples, where each column depicts the mean+SE for triplicate results of these samples and the dashed line indicates the threshold for positive EV-LAM signal as determined by ROC curve analyses.

FIG. 19B is a graph showing normalized intensity (N.I.) of LAM-EV AuR or Cu-NEI signal obtained for 5-fold serial dilutions of a TB-positive serum sample. Data depict Mean+SE results of triplicates.

FIG. 20A is a chart showing clinical results for the TB and non-TB groups, where the TB group is split into subgroups diagnosed by microbiological findings (culture or Xpert results; group A) or by at least two of the following criteria: symptoms suggestive of TB, positive anti-TB treatment (ATT) response, chest X-ray consistent with TB, or a TB contact or tuberculin skin test (TST) consistent with Mycobacterium tuberculosis infection.

FIG. 20B is a Venn diagram of TB case classifications by Cu-NEI, Mtb culture and Xpert results.

FIG. 20C is a table comparing sensitivity, specificity, and accuracy estimates, and 95% confidence interval (95% CI) values determined using the clinical and study test results and Mtb EV AuR and Cu-NEI results reported for the 31 pediatric patients shown in FIG. 20A.

### DETAILED DESCRIPTION

Method and assays of the disclosure utilize a Cu growth strategy that can increase the size and modifies the geometry of conventional gold nanostructures, such as gold nanorod (AuR) nanoparticles that exhibit excellent binding kinetics prior to crystalline Cu growth to maximize resulting localized surface plasmonic resonance (LSPR). The methods and assays of the disclosure can result in an average 71-fold increase in nanoparticle-induced signal intensity across a broad linear concentration range. Methods and assays of the disclosure can also beneficially enhance the signal from biomarker assays using nanoparticle probes. For example, an estimated 39 EVs/μL limit of detection was achieved when Cu growth was induced on antibody-conjugated Au nanoparticles bound to target biomarkers on extracellular vesicles (EVs).

Assays of the disclosure utilize a Cu growth approach that is capable of enhancing the single signal for nanoplasmonic enhanced immunoassays (Cu-NEI) to enhance scatting signals for extracellular vesicle detection. The assays of the disclosure can advantageously provide improved detection sensitivity in an easy to use process, thereby making it a clinically suitable process. The use of Cu growth can also provide colorimetric based detection, which can be useful in techniques such as paper strip lateral flow assays.

Copper reduction can be controlled to avoid overgrowth and non-specific reductions by interferants present in complex biological systems. Nanoparticle growth in seed-mediated outgrowth methods is largely determined by the crystal structure of the initial nanoparticle substrate. Two widely used gold substrates include gold nanorods (AuR) and gold nanospheres (AuS). AuR nanoparticles tend to exhibit monocrystalline structure since their geometry allows Au atoms to align along the length of their longest axis of symmetry, and zero-valent Cu atoms tend to align with this crystal structure to serve as a seed for monocrystalline Cu nanoshell growth. This crystalline structure is largely absent in AuS nanoparticles since their complete rotation symmetry does not favor regular alignment of their Au atoms. Selective area electron diffraction (SAED) images of the AuR and AuS particles selected for Cu nanoshell growth detected primarily monocrystalline and polycrystalline

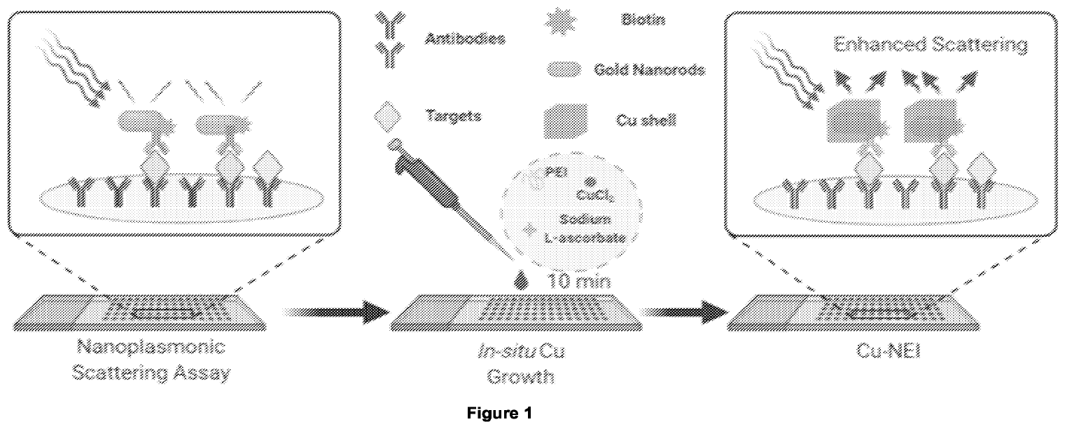

Referring to FIG. 1, in accordance with the disclosure, an assay can include contacting a substrate having a first capture antibody with a sample containing extracellular vesicles. The sample can include target and non-target extracellular vesicles. The capture antibody is selected to bind to all extracellular vesicles in the sample, thereby immobilizing the extracellular vesicles onto the substrate. The substrate having the immobilized extracellular vesicles is then contacted with gold nanostructures associated with a detection antibody. The detection antibody is a target-EV specific antibody, which binds only with the target extracellular vesicles to thereby selectively label the target extracellular vesicles with the gold nanostructures. The assay then further includes incubating the substrate in a copper growth reagent solution for site specific copper shell (Cu shell) growth on the gold nanostructures. During incubation, copper ions present in the reagent are reduced to metallic copper and selectively form a copper shell around the gold nanoparticles. Finally, the assay can include detecting a scattering signal from the sample after Cu shell growth. The Cu shell improves the scattering signal as compared to the signal from the gold nanostructures alone. This can enhance the detection sensitivity of the assay, particularly where the target extracellular vesicles are present in the sample in low concentrations.

The assay can include washing steps. For example, non-bound portions of the sample can be washed away from the substrate after immobilizing the extracellular vesicles to the substrate. Additional washing can be performed, for example, after labeling the target extracellular vesicles with the detection antibody and gold nanostructure to remove, for example, unbound nanostructure. Finally, a washing step can be performed after incubation with the copper growth reagent to remove and residual reagent before imaging and detection of the scattering signal. For example, after blocking, the substrate can be washed. Washing can be performed multiple times, for example 3 times. PBS can be used in the washing steps to prevent the vesical structure breaking. The substrate should be washed by distilled water to remove unbound particles and salt before they were subjected to DFM photography.

The sample can be any sample containing extracellular vesicles. For example, bodily fluids can be used as the sample, including, but not limited to, urine, blood, plasma, serums, saliva, milk, amniotic fluid, cerebrospinal fluid, synovial fluid, BALF, CSF, tears, sweat and the like.

The substrate can be any suitable substrate material. For example, the substrate can be a glass slide. For example, the substrate can be a multi-well plate. For example, the substrate can have a coating or film to define a multi-well surface on the substrate. For example, a multi-well PDMS film can be coated on a substrate such as glass to provide the glass substrate with the PDMS film defining the separators on the glass slide to divide the substrate surface into multiple wells. The substrate can be surface modified using any known surface modifications and modification techniques. For example, the substrate can be coated, for example, with an antibody binding coating. For example, the substrate can be coated with a protein A/G coating. For example, the substrate can be a glass surface with Silane which can immobilize capture antibodies. For example, the substrate can be an SLB surface.

The substrate is coated with a first capture antibody. Any suitable first capture antibody for capturing extracellular vesicles can be used. For example, CD81, CD63 or CD9 antibodies are well established common markers for extracellular vesicles that can immobilize extracellular vesicles on the surface.

For example, the capture antibody can be coated on the substrate and washed to remove unbound antibody. For example, washing 3 times with PBS has been found effective to remove unbound antibody. The concentration of capture antibody can be about 1 ug/mL-20 ug/mL. Substrates having an aminoalkylsilane surface and/or a Protein A/G surface can bind antibodies. However, use of Protein A/G has been observed to bind the antibodies in good orientation.

The substrate is contacted with the sample under conditions sufficient to bind the extracellular vesicles to the first capture antibody. For example, the sample can be incubated with the substrate containing the capture antibody at about 4° C. overnight or at about 20° C. to about 40° C. for about 0.5 hour to about 4 hours.

Optionally, the sample can be centrifuged before incubating with the substrate to remove contaminants. For example, the contaminants can be removed with the pellet after centrifugation and the supernatant can be incubated with the substrate. The sample can be centrifuged for example, for 30 mins to about 1.5 hours at 10,000 rpm.

The detection antibody can be selected by those skilled in the art depending on the target extracellular vesicle to be captured. The detection antibody is capable of interacting with biomarkers expressed on extracellular vesicle surface. For example, the detection antibody can be biotinylated to binder with an avidin-modified Au structure. The collected biomarker can facilitate disease diagnosis or progression detection. For example, the disease can be cancer or infectious disease.

The detection antibody can be present in an amount of about 1 μg/ml to about 5 μg/ml.

The substrate having the extracellular vesicles immobilized thereon and with the target extracellular vesicles labeled with the gold nanostructures is incubated in the copper growth reagent for a time sufficient to allow the copper ions present in the copper growth reagent to reduce and form a copper shell around the gold nanostructures. For example, the incubation time can be about 5 min to about 30 min, about 8 min to about 10 min, about 10 min to about 20 min. Other suitable incubations times include about 5, 6, 7, 8, 9, 10, 11, 12, 13, 14, 15, 16, 17, 18, 19, 20, 21, 22, 23, 24, 25, 26, 27, 28, 29, or 30 min and any values there-between or ranges defined between any such values.

The copper growth reagent includes copper ions (Cu²⁺), a structure-directing agent, and a reductant. The reductant is present in a concentration that is in excess as compared to the copper ion and structure-directing agent.

The structure directing agent mediates controlled nanostructure crystallization of the copper into the core-shell structures. In the presence of reductant, Cu⁰ outgrowth along with the structure directing agent provides for the formation of the nanoshell structure around the gold cores. The structure directing agent can be a branched polymer. The structure directing agent can be, for example, polyethyleneimine (PEI), poly(vinyl alcohol); polyacrylic acid (PAA), polyvinylpyrrolidone (PVP); poly-L-Lysine (PL)

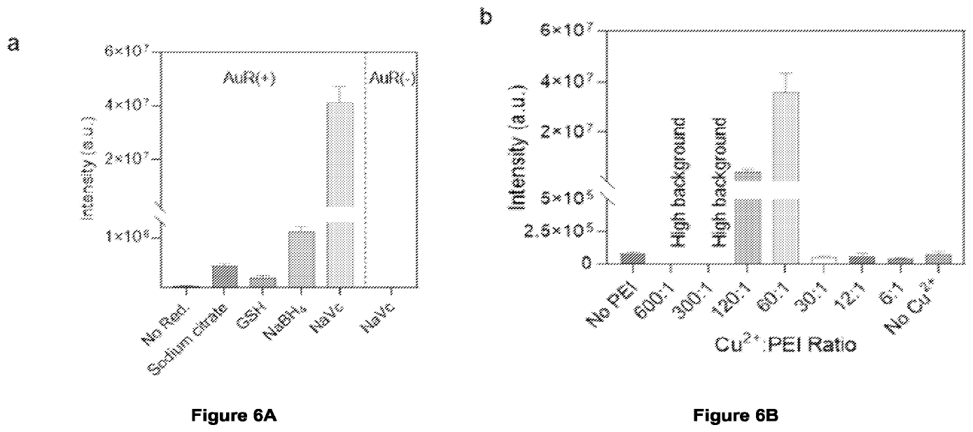

The ratio of the copper ions to the structure-directing agent can be about 120:1 to about 60:1, about 100:1 to about 80:1, about 95:1 to about 65:1, about 110:1 to about 70:1, about 105:1 to 75:1. Referring to FIG. 6B, it was observed that below a 60:1ratio, the structure-directing agent failed to chelate the copper ions to form nanostructures in a reductive environment, while a ratio above 120:1 caused unwanted high background on the surface protein A/G precoated slides.

The reductant can be or include ascorbic acid and/or citrate. For example, the reductant can be (+)-Sodium L-ascorbate (NaVc) In general, the reductant needs to be selected such that it can reduce the Cu ions to metallic Cu can be used for the Cu in-situ growth. Referring to FIG. 6A, various reductants were tested. The reducibility of sodium citrate and glutathione were too mild to reduce sufficient Cu shell within a suitable time and resulted in non-specific background on protein A/G precoated surfaces. NaBH₄ led to self-nucleation of the structure-directing agent-Cu complex that consumed most of the copper ions without seeding on the gold core, resulting in limited copper shell formation after washing. However, the harsh reductant environment of NaBH₄ could be harmful to the integrity of the lipid-bilayer structure of the extracellular vesicles. NaVc had moderate reducibility and was able to reduce copper ions into metallic copper on the surface of the gold core, while limiting the self-nucleation of the structure directing agent-Cu complex within a suitable incubation time. Sodium ascorbate was found to provide suitable reducibility at room temperature and within incubation times practical for clinical use.

A kit for performing an assay in accordance with the disclosure can include a substrate coated with a capture antibody, gold nanostructures having a detection antibody associated therewith, a copper growth reagent, and instructions for contacting the substrate with the sample to immobilize extracellular vesicles contained therein through binding with the capture antibody, labeling the sample with the gold nanostructures by binding the gold nanostructures to the target extracellular vesicles through binding with the detection antibody, and incubating the sample with the copper growth reagent to form copper shells around the gold nanostructures.

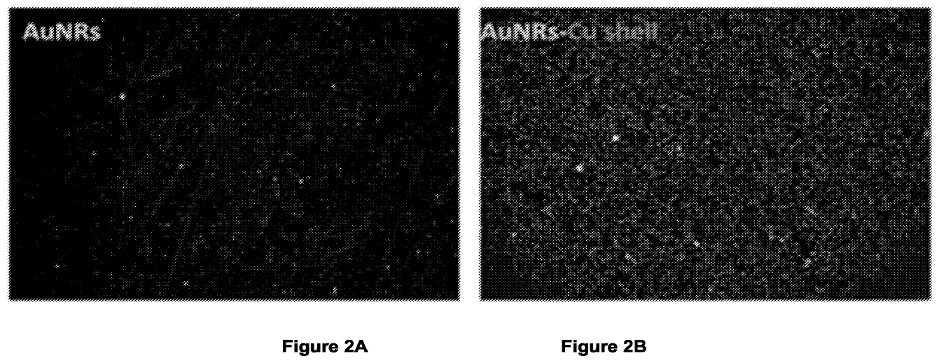

Referring to FIGS. 2A and 2B, a comparison of the dark field images before (FIG. 2A) and after (FIG. 2B) Cu growth illustrates the signal enhancement achieved with the assay of the disclosure. A 100-to-3000-fold increase in signal strength was observed with the Cu shell growth.

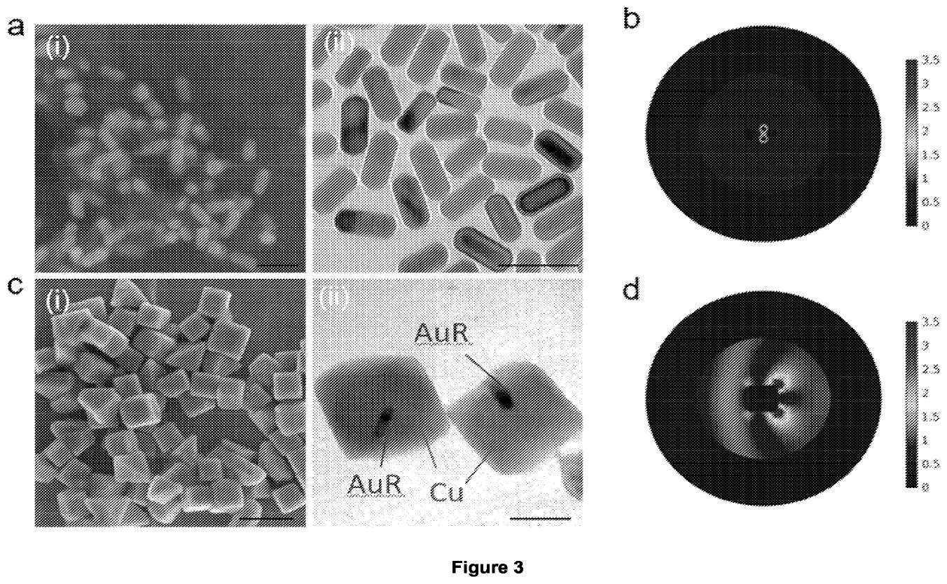

The shape and size of the gold nanorods was confirmed by scanning electron microscopy (SEM) and transmission electron microscopy (TEM) (FIG. 3A). Gold nanorods with dimension of 25 nm×71 nm showed an inherent surface plasmonic resonance (SPR) effect. COMSOL modeling was used to simulate SPR on the surface of the cold nanorods under electric field (FIG. 3B). Referring to FIG. 3C, SEM and TEM imaging showed that reduced copper formed large particles and the shape of the formed cooper particles was observed o be uniform cubical or tetrahedron shaped Cu shells. TEM imaging confirmed that the cubic Cu nanoshell formed around the gold nanorod core. Simulations of the SPR of the Cu-shell/gold nanorods using the COMSOL model indicated a strong refraction of the copper shell and the edges and corners of the cube (FIG. 3D).

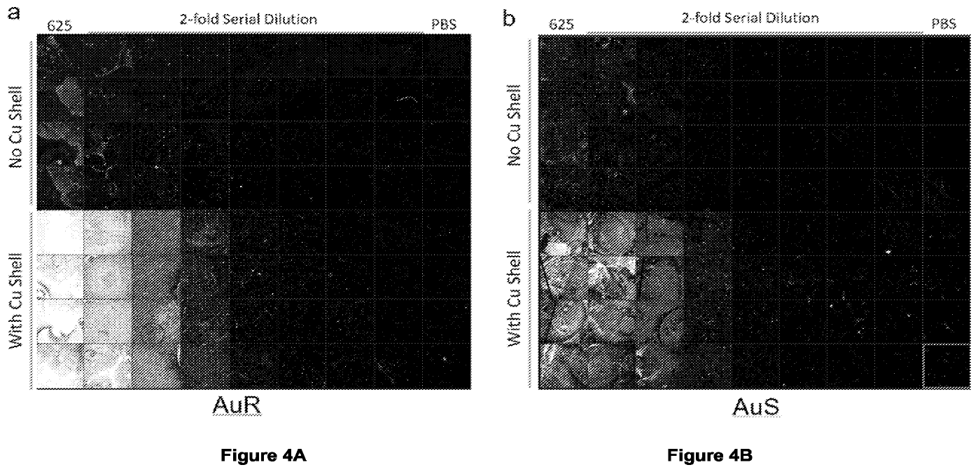

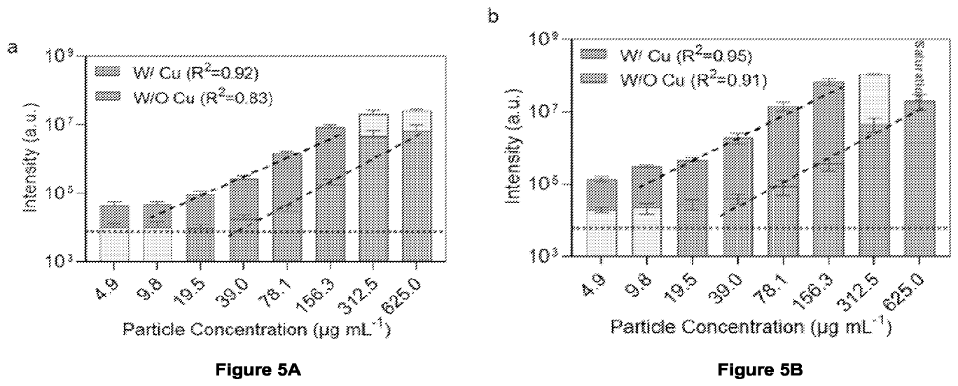

Referring to FIGS. 4A and 4B, the enhancement in scattering signal was further evaluated using dark field imaging. Antibody-coated gold nanorods and gold nanospheres were attached to a protein A/G functionalized slide through interaction of the antibodies with the protein A/G. A 2-fold serial dilution from 625-0 μg/ml was used along with covalent binding of the gold nanostructures in a gradient to evaluate the sensitivity enhancement achieved with the presence of the copper shell. Copper shells were grown on the gold nanostructures in accordance with the disclosure by incubating the slide having the gold nanostructures immobilized thereon in a copper growth reagent under conditions sufficient to reduce copper ions to metallic copper and form the copper shell structure. A significant enhancement of the signal was observed with the presence of the copper shell. Referring to FIGS. 5A and 5B, the scattering signal intensity of gold nanorods was enhanced 10² to 10³-fold with presence of the Cu shell, while the gold nanospheres had a 10-10² fold enhancement in the signal intensity. The signal enhancement of the copper shell-gold nanorods at higher concentrations of the gold nanorods resulted in over exposure of the CCD on DMF and therefore are not reported in FIG. 5A.

### EXAMPLES

### Example 1

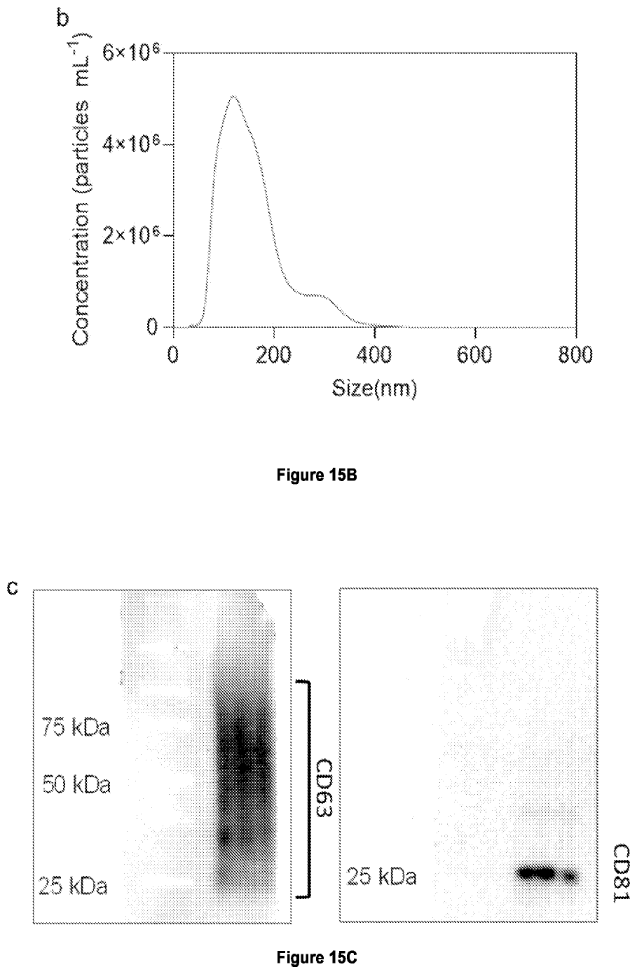

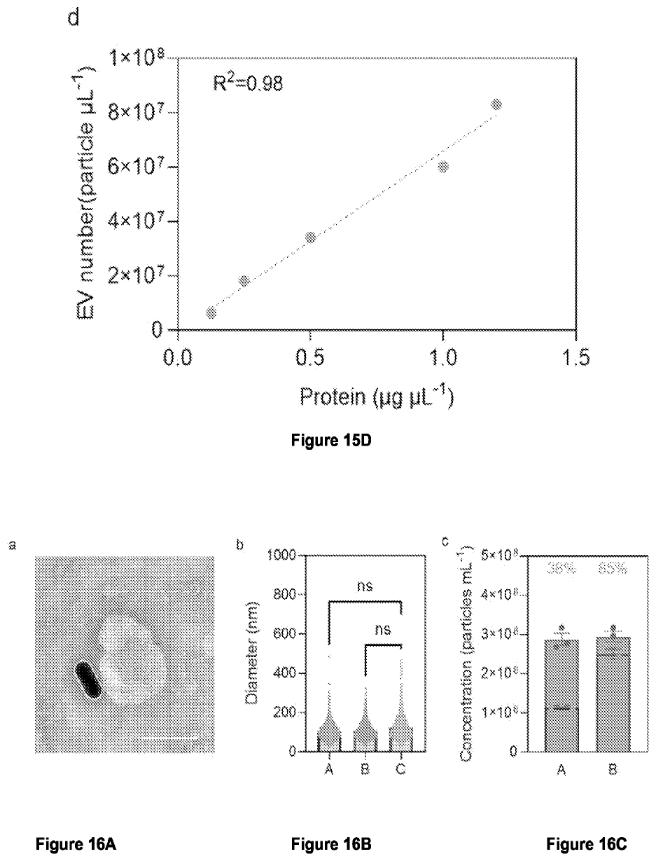

To evaluate the ability of the extracellular vesicles to tolerate AuR@Cu growth conditions, standard extracellular vesicles isolated from HCT-116 line (Novus Biologicals) were used. These EV fractions were observed to have the expected size distribution, CD63 and CD81 biomarker expression, and morphology expected for small EVs derived from this cell, and revealed a strong correlation between sample protein concentration and EV abundance determined by nanoparticle tracking analysis (NTA) data (FIG. 15). These EV samples were also found to bind AuR NPs conjugated with anti-CD63 antibodies. (FIG. 16A). To evaluate the potential of Cu shell growth conditions to disrupt antigen-antibody probe interactions or destabilize lipid bilayers to attenuate biomarker detection, we incubated these EVs with PBS or Cu nanoshell growth reagents+BSA-blocked AuR then centrifuged them to remove AuR@Cu or self-nucleated Cu NPs and analyzed the EV supernatant by NTA to evaluate potential changes in EV diameter or abundance. EV in all these samples exhibited similar size distributions (FIG. 16B), although fewer EVs were detected in the Cu growth reactions, which we hypothesized was due to non-specific interaction and precipitation of EVs by the AuR@Cu NPs or self-nucleated CuNPs generated in these samples. This conclusion was supported by the observation that that 85% and 38% of input EVs were recovered after centrifugation of EV samples incubated with BSA-blocked versus unblocked AuR@Cu samples (FIG. 16C), indicating that Cu growth buffer conditions did not disrupt EV integrity.

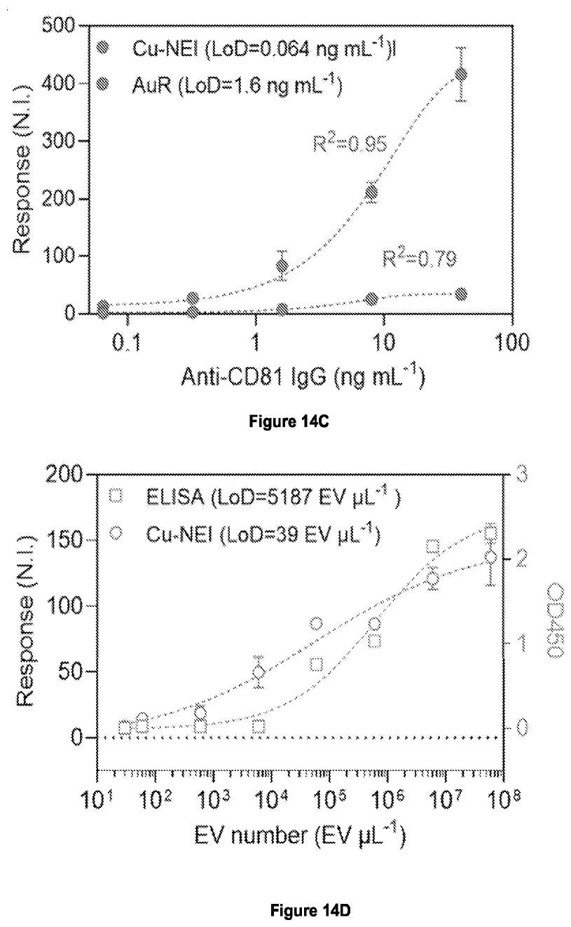

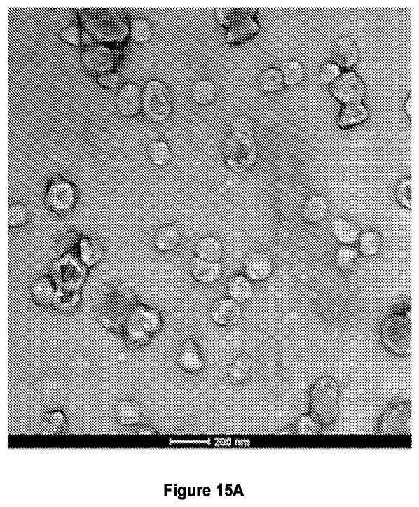

In this assay to test the ability of Cu-NEI to detect an EV surface biomarker, EVs standards of known concentration were spiked into an EV-depleted fetal bovine serum matrix, then captured on a detection slide pre-coated with an antibody to the EV surface protein CD81, incubated with a biotinylated antibody specific for the EV surface protein CD63, and then washed and hybridized with avidin-conjugated AuR or horseradish peroxidase probes. Cu-NEI and ELISA LODs determined in this analysis (FIG. 14D were 39 and 5187 EVs μL⁻¹, respectively, while the signal detected from the AuR probe prior to Cu nanoshell growth yielded a LOD of 800 EVs μL⁻¹ (FIG. 16A). Cu-NEI EV assay was thus approximately 20-fold and 130-fold more sensitive than the AuR and ELISA methods, respectively, and also revealed a linear range that was about 2 orders of magnitude wider than that of the EV ELISA.

These results suggest that AuR@Cu growth on captured EVs did not disrupt these EVs, although it is possible that interaction of growing AuR@Cu crystals with the assay plate during the Cu growth reaction could prevent their loss in the event of EV lysis. To address this possibility, captured EVs were incubated with or without a non-denaturing lysis solution before or after the AuR@Cu growth reaction, and found that pre-addition of the lysis buffer completely ablated Cu-NEI assay signal when samples were BSA-blocked prior to the Cu growth reaction, and that EV lysis after Cu growth reaction had a similar albeit lesser effect (FIG. 16B, implying that EV integrity is required to generate and retain Cu-NEI signal on captured EVs

### Example 2

To test exosome detection of the assay of the disclosure, standard exosomes derived from HCT-116 line (Novus Biologicals) with CD81 and CD63 as the capture antibody and the detection antibody, respectively, were used. Standard extracellular vesicles were spiked into exosome-depleted human serum. The concentration of extracellular vesicles ranged from 30×10⁷ particles/μL to 6×10⁷ particles/μL. The sample was processed using the assay of the disclosure and a standard ELISA procedure using the same capture and detection antibodies for comparison.

The assay of the disclosure was performed using a protein A/G coated substrate having the CD81 capture antibody coated thereon. The substrate was a multi-well slide, with the surface of each well coated with protein A/G. The sample was then dropped onto the substrate to immobilize the extracellular vesicles through interaction of the extracellular vesicles with the capture antibody. Gold nanorods modified with the CD63 detection antibody were added to label the target extracellular vesicles with the gold nanorods through binding of the detection antibody and the extracellular vesicles. All extracellular vesicles expressing the CD63 biomarker were labeled with the CD63 antibody-modified gold nanorods. The substrate was then incubated in a copper growth reagent containing Cu²⁺ ions and PEI in a ratio of 60:1, along with sodium ascorbate as the reductant. The substrate was incubated in the copper growth reagent for 10 min to allow for copper shell growth.

For comparison, ELISA was performed using the same capture and detection antibodies according to standard procedures known in the art.

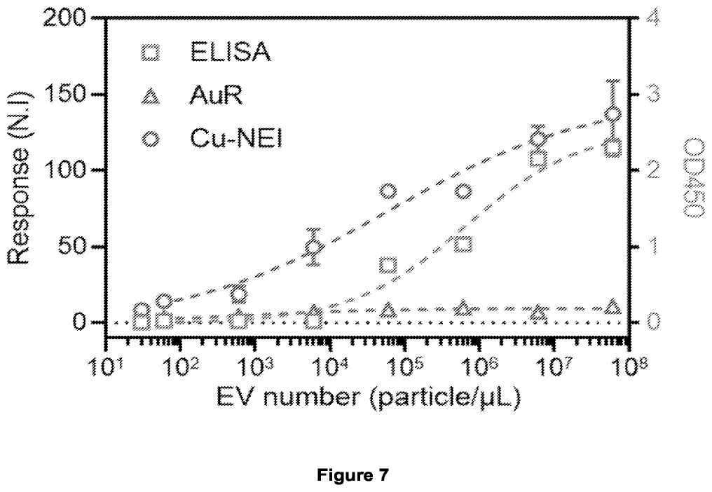

As shown in FIG. 7, the assay of the disclosure was capable of detecting extracellular vesicles present in a concentration as low as about 1.8×10³ particles/μL, which was 100-fold lower than ELSA using the same capture and detection antibodies. Further, the dynamic range of extracellular vesicle detection of the assay of the disclosure was much sider than traditional ELISA.

### Example 3

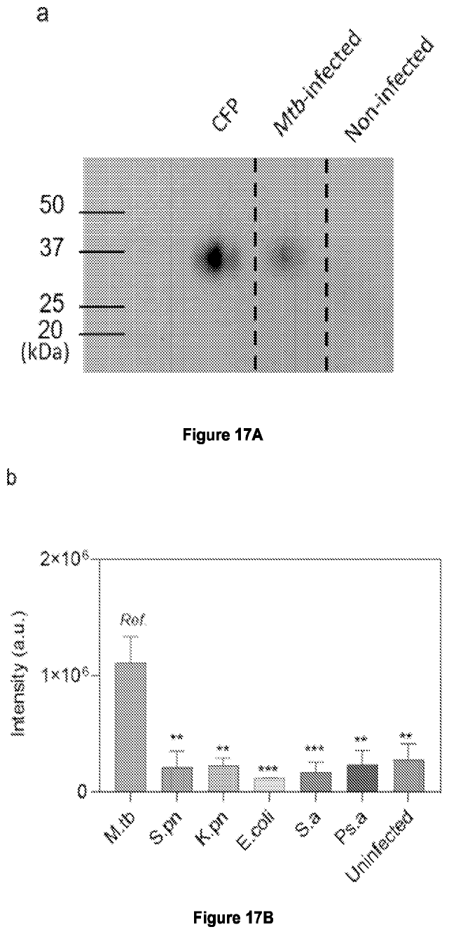

Diagnostic performance of an assay in accordance with the disclosure was tested with tuberculosis (TB) specific extracellular vesicles from a TB cohort with 21 positive (including 5 confirmed TB and 16 unconfirmed TB) and 10 negative cases (or unlikely TB). The patient population was a diagnostically challenging cohort of children living with HIV at high risk for TB-related morbidity. Glycolipid lipoarabinomannan (LAM) located on Mtb and associate its virulence and can located on the membrane of macrophage and circulating in peripheral blood. Those Mtb specific extracellular vesicles enable the diagnosis of TB or non-TB by targeting LAM on the membrane of extracellular vesicles. In some positive cases, the signal could be weak due to low amounts of circulating TB-specific extracellular vesicles. The diagnostic ability of a Cu-NEI assay in accordance with the disclosure was evaluated by its ability to detect Mtb glycolipid biomarker lipoarabinomannan (LAM) (FIG. 17A) on the surface of serum EVs derived from individuals with and without TB. LAM is reported to be highly enriched on EV membranes, and the detection of LAM on EVs isolated from different body fluids can be used to diagnose TB. Notably, the anti-LAM antibody employed in this study targets an Mtb-specific LAM motif, and should thus provide sufficient diagnostic specificity when used as the detection antibody in our serum-based Cu-NEI EV assay for TB diagnosis.

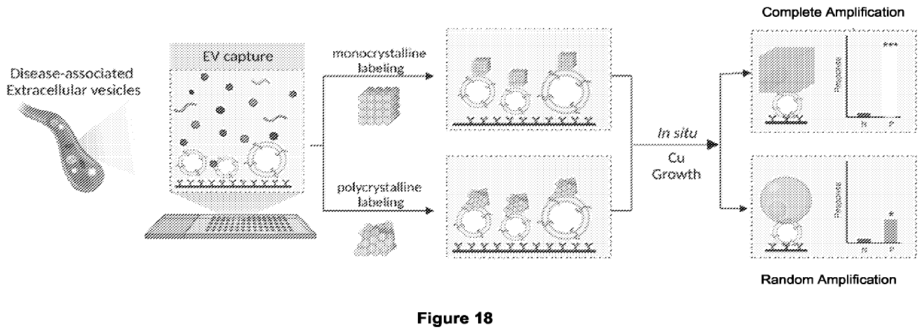

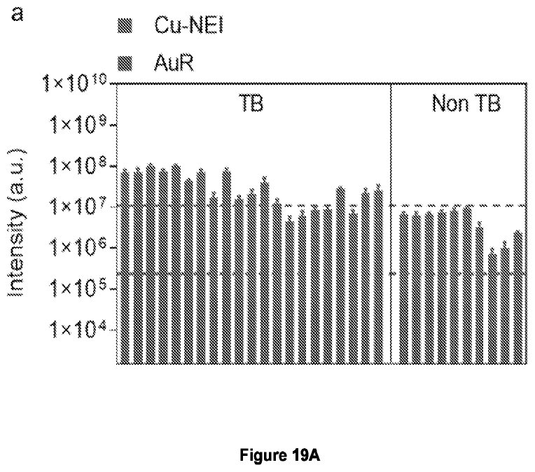

An assay in accordance with the disclosure was performed by as described in Example 2, except TB serum samples were used as opposed to the extracellular vesicle-spike exosome-depleted human serum. Referring to FIG. 18A, this assay directly captures EVs from serum to allow their direct incubation with a LAM-specific AuR probe that serves as a substrate for AuR@Cu nanoshell growth used to enhance the detection of low abundance LAM-positive EV signal.

This Cu-NEI method differentiated EVs released by Mtb-infected macrophages from those produced by equivalent cultures infected with five other common human pathogens, including several that cause respiratory infections (FIG. 17B). Therefore, this potential TB diagnostic approach was used to analyze serum samples obtained from a cohort of HIV-positive young children aged 0.2 to 9.7 years, as new diagnostic methods are needed to improve TB diagnosis in children, particularly HIV affected children, as conventional TB diagnostic methods demonstrate reduced performance in these populations. These children were thus classified as TB cases by microbial findings (confirmed TB), or algorithm that segregated them into unconfirmed TB or non-TB groups on the basis of other clinical data (Table 1).

TABLE 1
Classification criteria for pediatric
TB cases (adapted from Graham 2015)¹

Bacteriologic confirmed TB (Confirmed TB)
Positive microbiological culture or Xpert Mtb/RIF from respiratory
specimen
Clinician diagnosed TB (Unconfirmed TB)
Not bacteriologic confirmed but meet at least 2 of the following:
Symptoms features of TB
Expert panel findings for a TB-related death
Weight loss/Failure to thrive (unexplained weight loss, >5%
weight reduction in last 3 months, deviation from growth
trajectory, crossing of percentile lines in prior 3 months,
WAZ <=−2 or WHZ <=−2 in absence of prior growth trajectory AND
not responding to nutritional rehabilitation or ART if HIV+)
Persistent and unexplained fever (fever >1 week reported
by guardian, or objectively recorded at least once (>38.0)
Persistent and unexplained lethargy or decreased playfulness
reported by caregiver
Infants 0-16 days: neonatal pneumonia, unexplained
hepatosplenomegaly, or sepsis
CXR consistent with TB
Read by 2 independent, blinded reviewers; Quality of CXR indicated;
Standardized forms with predetermined terminology and yes/no options
for CXR reader.
Example criteria from South African Tuberculosis Vaccine Initiative
(SATVI):
1) Tracheal displacement or airway compression
2) lymphadenopathy suggested by soft tissue density
3) Air space opacification
4) Nodular picture (miliary or larger, widespread, and bilateral)
5) Pleural effusion
6) Cavities
7) Calcified parenchyma (Ghon focus)
8) Vertebral spondylitis
Close TB contact immunological evidence of Mtb infection
(TST or IGRA)
Positive response to TB treatment (clinical features suggestive of TB at
baseline have improved and no new clinical feature suggestive of TB)
Expert panel findings for a TB-related death
Unlikely TB (Non-TB)
Bacteriologic confirmations are NOT obtained, criteria for
unconfirmed TB are NOT met

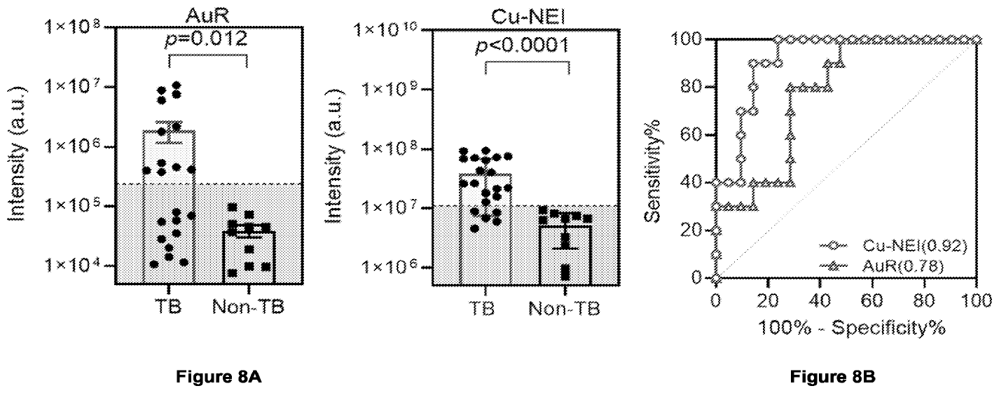

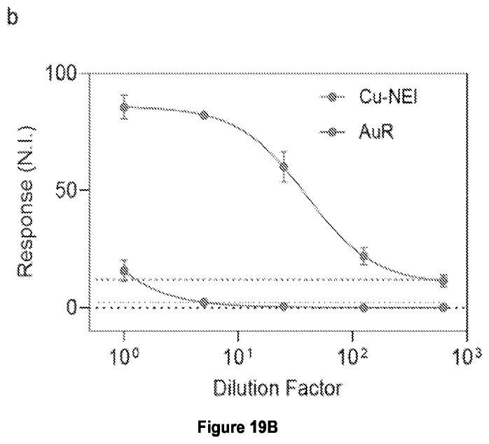

It was observed that serum EV LAM expression significantly differed when analyzed by either AuR immunoassay or Cu-NEI, although Cu-NEI signal was more intense and had less overlap among individuals with and without TB (confirmed and unconfirmed TB versus non-TB) to improve discrimination of their samples (FIGS. 8A and 8B and 19A) when using thresholds determined by their receiver operation characteristic (ROC) curve analyses (FIG. 8D). FIGS. 8A and 8B depict mean+SE values, where individual data points indicate the mean of three technical replicates, and p-values were calculated using two-tailed Mann-Whitney tests. Dashed line indicates the cut-off values for positive LAM-EV signal as determined by the receiver operation characteristic (ROC) curve analyses of the AuR and Cu-NEI data shown (FIG. 14B). The ROC curve data produced area under the curve values of 0.78 and 0.92 for the ability of the AuR and Cu-NEI results to distinguish these groups, much higher than the values determined using the corresponding Xpert and urinary LAM data (0.60 and 0.51).

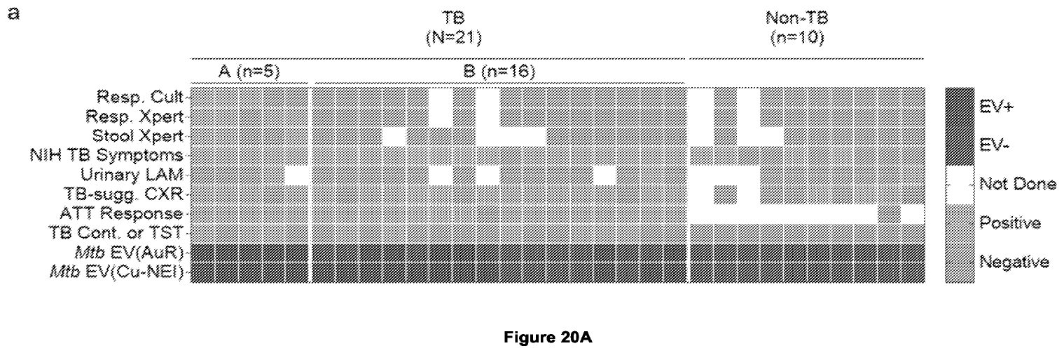

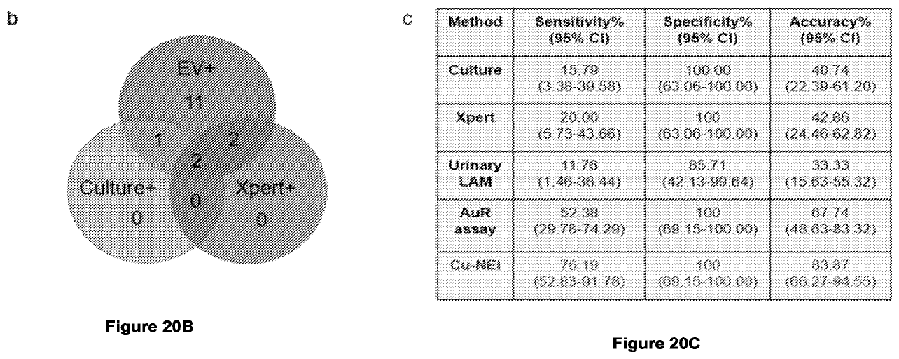

Comparison of AuR and Cu-NEI results with results NIH criteria for pediatric TB diagnosis did not reveal obvious associations between these two sets of data. All microbiologically confirmed TB cases revealed positive serum EV LAM results when analyzed by AuR and Cu-NEI assays, while respiratory culture and Xpert results were positive for three and four of these five cases, respectively (FIG. 20A, group A). However, fewer positive serum EV LAM results were detected for the clinically diagnosed TB group, with that AuR assay producing fewer positive results than Cu-NEI, and both assays detecting a lower fraction of clinically diagnosed (38% versus 69% sensitivity; 6/16 and 11/16; FIG. 20A, group B) than microbiologically confirmed TB cases, with both differences likely reflecting reduced EV LAM abundance in the clinically diagnosed group. However, EV LAM positive results detected by AuR assay and Cu-NEI were much more frequent than urine LAM assay positive results in both these groups for individuals with available results (1/4 and 1/13). Notably, the urine LAM positive rates observed in these two TB groups were similar to the positive rate observed in the non-TB group (1/7), emphasizing the poor diagnostic utility of this test. No obvious pattern of strong agreement between positive Cu-NEI results and individuals with TB-associated symptoms (10 of 15 match) or CXR finding (9 of 12 match).

Cu-NEI positive results for serum EV LAM expression detected 15 of the 21 TB cases in this cohort, including all the microbiologically diagnosed TB cases and the majority (69%; 11 of 16) of its clinically diagnosed TB cases (FIG. 20B), and thus can directly detect a disease group missed by other diagnostic tests and thus exhibits greater overall diagnostic performance than other direct tests (FIG. 20C). The ability of the Cu-NEI assay to rapidly a detect low abundance serum EV biomarker to enable disease diagnosis has significant implications for TB disease control, and potentially other diseases where similar EV biomarkers may circulate at low levels. In the current example, the diagnosis of pediatric TB cases can take weeks to months if not diagnosable by the PCR-based test such as Xpert, since Mtb culture can require up to 8 weeks to provide a final result,^{[36]} while criteria used in aggregate for clinical diagnosis can also require weeks (symptom duration) or months (positive response to anti-TB treatment) and be difficult to evaluate in conjunction with other infections or complications. The preeminent performance of Cu-NEI over AuR assay also can be verified by checking the LAM-EV intensity after serial dilution (FIG. 19B). One serum sample with serially diluted with strong LAM-EV expression, intensity of AuR assay decreased to 15% of its initial intensity after 5-folds dilution, but for Cu-NEI assay, the intensity of 125-folds diluted sample still retains up to 70%, demonstrated that Cu-NEI amplified signals from low-level EVs, enable better diagnosis performance on EV-based analysis.

### Example 4

The assay of the disclosure also demonstrated improved performance in predicting treatment response after immunotherapy as compared to PD_L1 biopsy score and ELISA results. Extracellular vesicles associated with PD-L1 quantification was performed to evaluate the correlation to immune-treatment patients in a non-small cell lung cancer study. The assay of the disclosure was performed as described in Example 1, using clinical serum samples from non-small cell lung cancer patients as the sample.

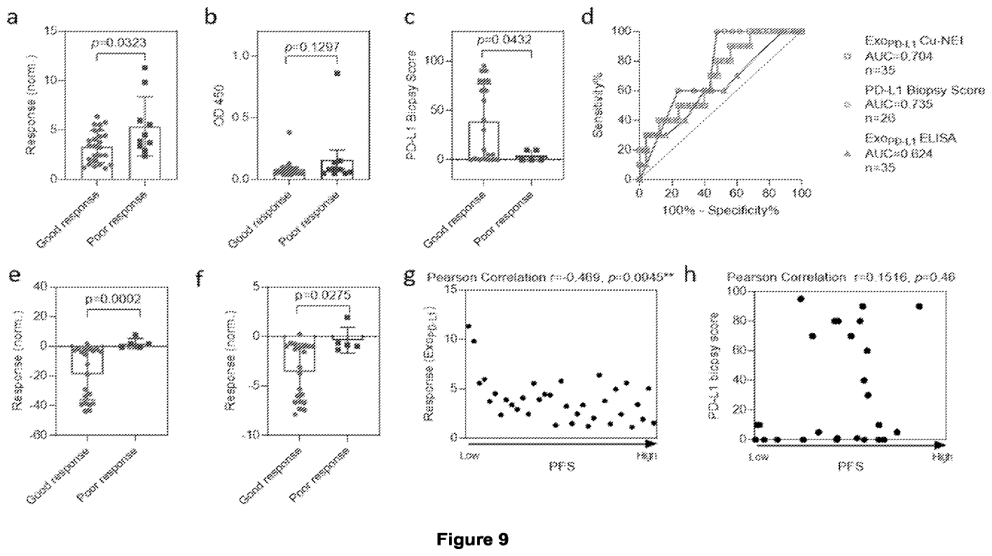

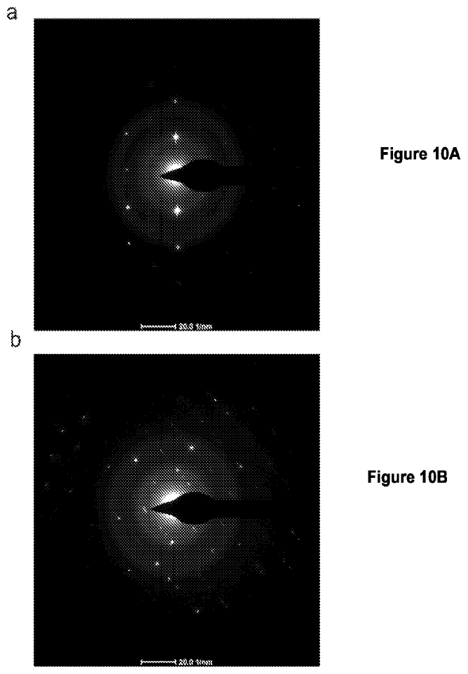

The combination of ExoPD-L1 with biopsy score was able to different the good response group from the poor response group (FIG. 9C) and was ultimately better than the combination of ELISA and biopsy score (FIG. 5F). ExoPD-L1 detected by the assay of the disclosure and progression free survival (PFS) time correlated negatively (FIG. 9G), while biopsy score and PFS were barely correlated (FIG. 9H). This further demonstrates that the assay of the disclosure provides a stable and robust performance in extracellular vesicle analysis and could be used to predict treatment response after PD1/PD-L1 immunotherapy.

### Example 5

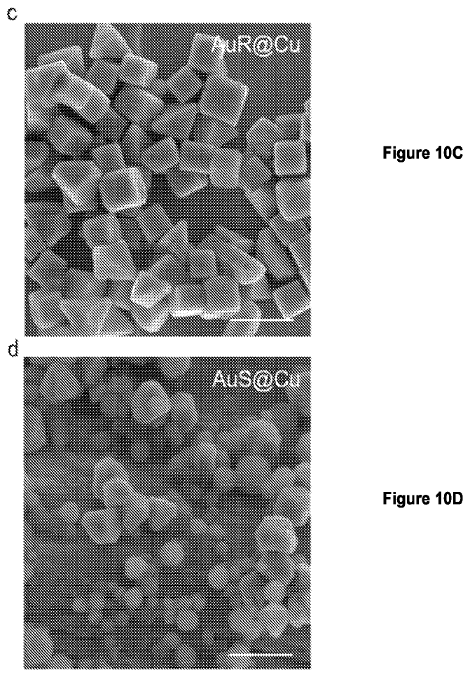

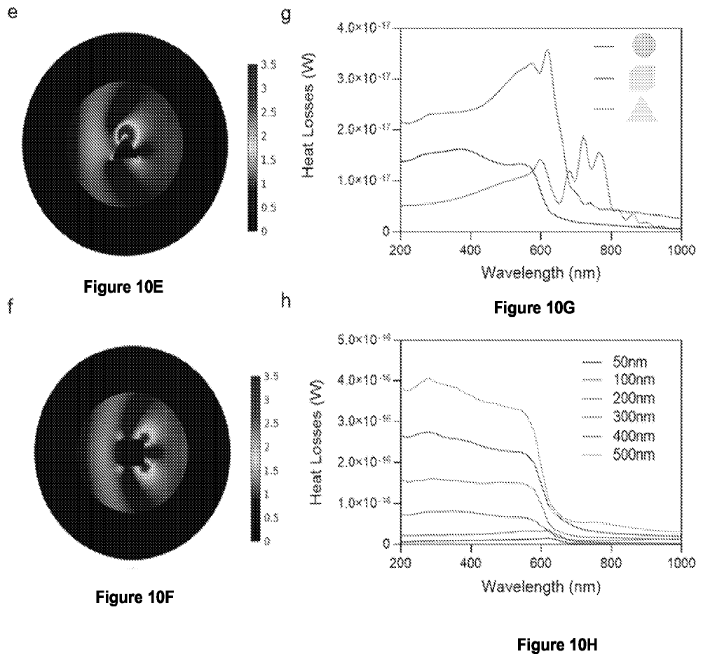

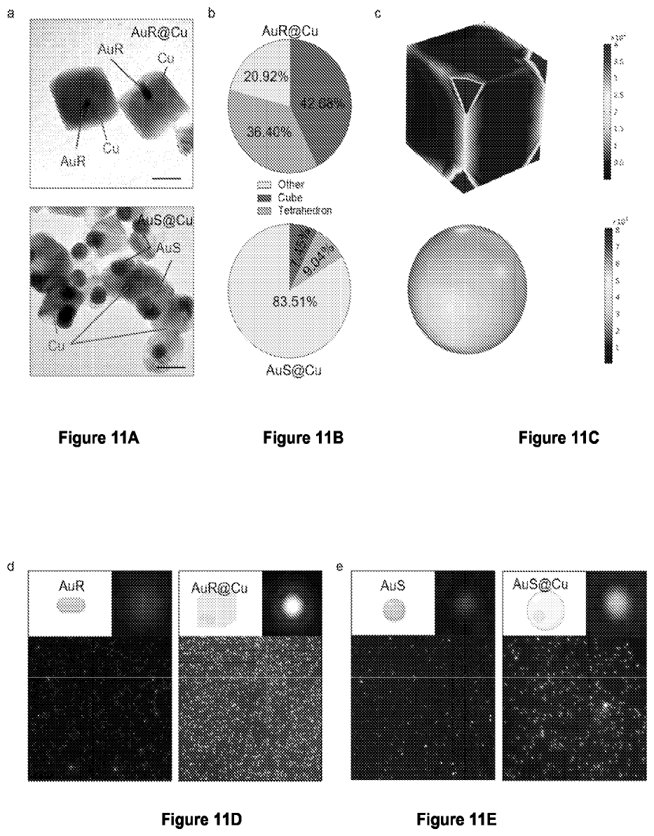

To induce Cu nanoshell growth AuR and AuS NPs were incubated with a CuCl₂ solution containing polyethyleneimine (PEI) as a structure-directing agent to promote controlled Cu deposition and the sodium salt of Vitamin C (NaVc) as a reducing agent, and the resulting NPs were then analyzed by transmission electron microscopy (TEM) and scanning electron microscopy (SEM) to evaluate the relative percentage of nanoshell structures produced in these reactions (FIGS. 11A and 11B and FIGS. 10C and 10D). Cu nanoshell reactions that used an AuR substrate produced primarily cubic and tetrahedral NPs (˜79%), in agreement with the common geometry of monocrystalline Cu NPs, while reactions that employed an AuS substrate primarily produced asymmetric NPs that lacked defined faces and edges (˜84%) (FIGS. 11A and 11B), with similar results detected in SEM images of these NPs (FIGS. 10C and 10D). The edges on cubic and tetrahedral NPs should exhibit much stronger plasmonic scattering activity, as predicted by Mie theory, and thus the Cu-coated AuR nanoshell NPs (AuR@Cu) would be expected to produce more intense plasmonic signal than the corresponding Cu-coated AuS nanoshell NPs (AuS@Cu). This agreed with the results we observed when the scattering of a plane wave of incident light from AuR@Cu and Aus@Cu was modeled using nanocubes and nanospheres of the same size (200 nm edge length or sphere diameter) based on Mie theory (FIG. 11C), where these shapes were chosen to reflect the regular geometry versus amorphous shapes of the NPs produced in these reactions. Maximum dissipated loss from this nanocubes was 5-fold greater than observed for the nanosphere (40000 W/m³ vs. 8000 W/m³). Similar strong plasmonic scattering was observed on the edges and corners of a corresponding tetrahedron with edge length matching the cube (FIGS. 10E and 10F). Simulation of the heat loss of these three NP shapes in the visible spectrum (380 nm-700 nm) as an indirect means to model their overall scattering activity (FIG. 10G) found heat losses progressively decreased from the cubic to tetrahedral to spherical NPs (6.2 vs. 3.0 vs. 2.1×10⁻¹⁷ W per NP). A similar analysis also found that heat loss increased with NP size (FIG. 10H). This scattering difference was manifest in AuR NPs analyzed before and after in situ AuR@Cu growth (FIG. 11D). Correspondingly, AuS@Cu NPs generated a comparatively weaker scattering signal under the same imaging conditions (50 ms exposure time) (FIG. 11E).

### Example 6

The effect of different reductants on monocrystalline Cu nanoshell growth was

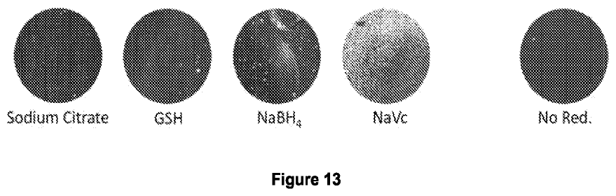

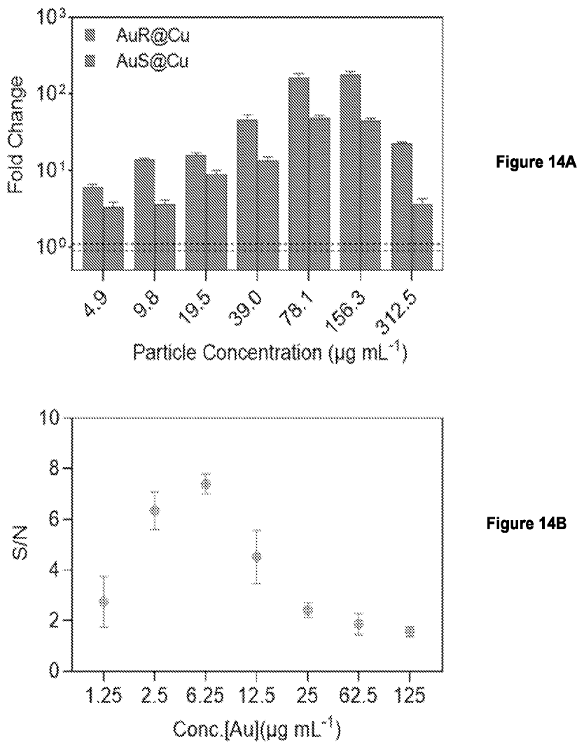

analyzed by varying the reductant employed in reactions containing constant amounts of AuR and Cu growth solutions, and the structure-directing agent polyethyleneimine (PEI). Glutathione (GSH), sodium citrate, and sodium borohydride (NaBH₄) had modest effects on Cu nanoshell deposition at room temperature within certain time, while NaVc produced a signal enhancement effect that was about 10-fold greater than detected with any of the other reductants (FIG. 6A). Without intending to be bound by theory, it is believed that NaBH₄ modestly increased scattering that may be caused by the formation of numerous insoluble Cu seeds that consume the CuCl₂ solution to decrease AuR@Cu formation and may also produce a harsh reductant environment that could disrupt protein-protein interactions or lipid bilayer stability. By contrast, the moderate reductant activity of NaVc reduced Cu²⁺ ions to metallic Cu on the AuR cores, without inducing significant self-nucleation of PEI-Cu complexes in the absence of AuR (FIGS. 6A and 13).

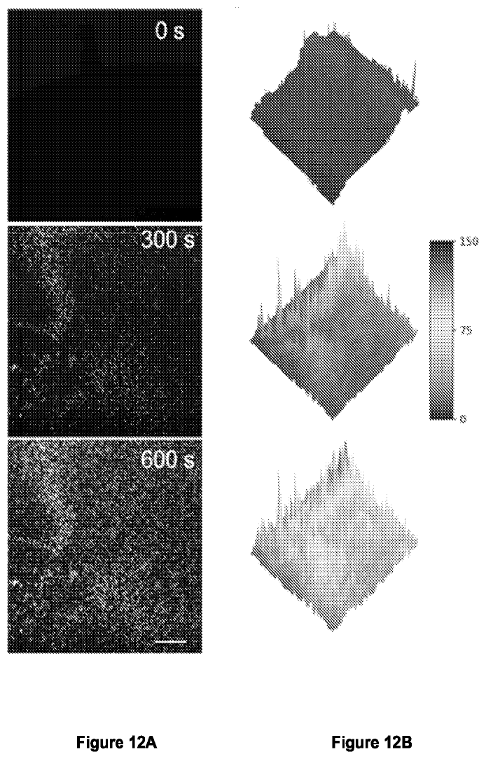

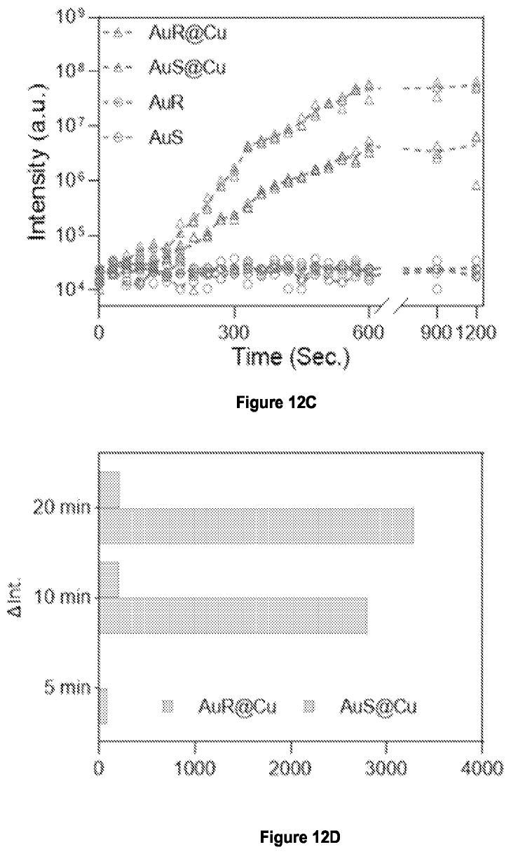

Referring to FIG. 6B, the effect of adding variable amounts of PEI to constant concentrations of CuCl₂ and NaVc on AuR@Cu growth. Amounts of Cu²⁺: PEI ranging from 600:1 to 6:1 were tested. It was observed that plasmonic signal intensity peaked at a 60:1 CuCl₂:PEI molar ratio, and did not markedly differ in reactions that lacked either PEI or CuCl₂ or had CuCl₂:PEI ratios≤30:1, but that background increased at CuCl₂:PEI ratios≥300:1, as increased signal detected in these samples was detected in the absence of the AuR substrate. AuR@Cu formation, as detected by scattering, rapidly occurred at room temperature in reactions using these optimized conditions (FIGS. 12A and 12B). Comparison of plasmonic scattering intensity over time from AuS and AuR probes treated with and without Cu nanoshell growth reagents (FIG. 12C) found that AuS and AuR signal remained constant over time, while AuR@Cu signal increased more rapidly (4.8×10⁶ vs. 4.3×10⁵ a.u./min) and plateaued higher than AuS@Cu signal (4.8×10⁷ vs. 4.3×10⁶ a.u.). Notably, AuR@Cu signal was 2.8×10³-fold greater than AuR signal after 10 minutes growth and increased only 1.7-fold more after a 20-minute total incubation period, while AuS@Cu signal was 200-fold greater than AuS signal upon reaching a plateau after a 10-minute incubation period (FIG. 12D).

### Example 7

Strong correlations between NP concentration and scattering were detected for both AuS and AuR NPs over broad concentration ranges both with and without Cu growth, although nanoshell growth shifted these correlations to lower concentrations and improved their linearity (FIGS. 5A and 5B). However, scattering increases were consistently greater and more reproducible when AuR NPs were used as the Cu growth substrate, which was reflected in the limit of detection (LOD) estimated for the resulting AuR@Cu and AuS@Cu NPs (1.9 versus 5.2 μg mL⁻¹). This difference in signal increase was more obvious when Cu nanoshell signal was normalized to the signal produced by its NP substrate at each concentration (FIG. 14A).

To determine the AuR probe concentration required to produce the greatest Cu-NEI signal-to-noise ratio, biotinylated IgG were incubated on protein A/G precoated multi-well slides to allow the binding of serial diluted streptavidin conjugated AuR probe followed by Cu shell growth signal amplification. The added AuR probe concentration that yielded the strongest and most reproducible signal (6.25 μg mL⁻¹) was used for all future analyses. Serial dilutions of biotinylated IgG were then incubated with optimized AuR concentration to evaluate the signal difference observed with and without a Cu shell amplification step (FIG. 14C), which estimated LOD values of 0.064 ng mL⁻¹ and 1.6 ng mL⁻¹, respectively, for the Cu-NEI and AuR assays.

Modifications and other embodiments disclosed herein will come to mind to one skilled in the art to which the disclosed compositions and methods pertain having the benefit of the teachings presented in the foregoing descriptions. Therefore, it is to be understood that the disclosures are not to be limited to the specific embodiments disclosed and that modifications and other embodiments are intended to be included within the scope of the appended claims. Although specific terms are employed herein, they are used in a generic and descriptive sense only and not for purposes of limitation.

It is also to be understood that the terminology used herein is for the purpose of describing particular aspects only and is not intended to be limiting. As used in the specification and in the claims, the term “comprising” can include the aspect of “consisting of.” Unless defined otherwise, all technical and scientific terms used herein have the same meaning as commonly understood by one of ordinary skill in the art to which the disclosed compositions and methods belong. In this specification and in the claims which follow, reference will be made to a number of terms which shall be defined herein.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

The use of the terms “a,” “an,” “the,” and similar referents in the context of the disclosure herein (especially in the context of the claims) are to be construed to cover both the singular and the plural, unless otherwise indicated. Recitation of ranges of values herein merely are intended to serve as a shorthand method of referring individually to each separate value falling within the range, unless otherwise indicated herein, and each separate value is incorporated into the specification as if it were individually recited herein. The use of any and all examples, or exemplary language (e.g., “such as”) provided herein, is intended to better illustrate the disclosure herein and is not a limitation on the scope of the disclosure herein unless otherwise indicated. No language in the specification should be construed as indicating any non-claimed element as essential to the practice of the disclosure herein.

1. An assay for detecting target extracellular vesicles in a sample comprising or suspected to be comprising target and non-target extracellular vesicles, comprising:
providing a substrate having a capture antibody coated thereon, the capture antibody capable of binding with the target and non-target extracellular vesicles;
contacting the substrate with the sample under conditions sufficient to allow for binding of the target and non-target extracellular vesicles with the capture antibody, thereby immobilizing the target and non-target extracellular vesicles on the substrate;
contacting the substrate having the immobilized target and non-target extracellular vesicles with a labeling reagent comprising gold nanostructures associated with a detection antibody, the detection antibody being capable of binding only to the target extracellular vesicles, wherein upon binding of the detection antibody with the target extracellular vesicles, the target extracellular vesicles are labeled with the gold nanostructures;
incubating the substrate comprising the labeled target extracellular vesicles with a copper growth reagent under conditions sufficient to induce copper ions to reduce and form a copper shell surrounding the gold nanostructure, the copper growth reagent comprising copper ions, a reductant, and a structure directing agent; and
detecting a scattering signal from the gold nanostructures having the copper shell formed thereon.

2. The assay of claim 1, wherein the gold nanostructure comprises gold nanorods and/or gold nanospheres.

3. The assay of claim 1, wherein the substrate further comprises a coating of protein A/G for binding the capture antibody to the substrate.

4. The assay of claim 1, wherein the sample comprises one or more bodily fluids.

5. The assay of claim 4, wherein the one or more bodily fluids are selected from the group consisting of urine, blood, plasma, BALF, CSF, tear, sweat, and combinations thereof.

6. The assay of claim 1, wherein the substrate is a multi-well plate or comprises a multi-welled PDMS film.

7. The assay of claim 1, wherein the reductant is sodium ascorbate.

8. The assay of claim 1, wherein the structure-directing agent is polyethyleneimine, optionally wherein the polyethyleneimine is branched.

9. (canceled)

10. The assay of claim 1, wherein the ratio of Cu ions to structure-directing agent in the copper growth reagent is 120:1 to 60:1

11. The assay of claim 10, wherein the structure directing agent is polyethyleneimine (PEI), the copper ions are Cu²⁺, and the ratio of Cu²⁺ to PEI is 60:1

12. The assay of claim 1, wherein the substrate is incubated in the copper growth reagent for about 5 to about 30 min.

13. (canceled)

14. The assay of claim 1, further comprising washing the substrate after one or more of:
immobilizing the target and non-target extracellular vesicles on the substrate,
labeling the target extracellular vesicles with the label, and
incubating the substrate in the copper growth reagent.

15. (canceled)

16. (canceled)

17. A kit for performing an assay for detecting target extracellular vesicles in a sample comprising or suspected to be comprising target and non-target extracellular vesicles, the kit comprising:
a substrate coated with a capture antibody, the capture antibody capable of binding with the target and non-target extracellular vesicles;
a labeling reagent comprising gold nanostructures associated with a detection antibody;
a copper growth reagent, the copper growth reagent comprising copper ions, a reductant, and a structure directing agent; and
instructions for performing the assay comprising contacting the substrate coated with the capture antibody with the sample to immobilize the target and non-target extracellular vesicles on the substrate; contacting the immobilized target and non-target extracellular vesicles with the labeling reagent comprising the gold nanostructure associated with the detection antibody, whereby the detection antibody selectively binds with the target extracellular vesicles to thereby label the target extracellular vesicles, and incubating the substrate comprising the labeled target extracellular vesicles in the copper growth solution to form a copper shell around the gold nanostructure, and detecting a scattering signal from the gold nanostructures having the copper shell formed thereon.

18. The kit of claim 17, wherein the gold nanostructure comprises gold nanorods and/or gold nanospheres.

19. The kit of claim 17, wherein the substrate further comprises a coating of protein A/G for binding the capture antibody to the substrate.

20. The kit of any one of claim 17, wherein the sample comprises one or more bodily fluids.

21. (canceled)

22. (canceled)

23. The kit of claim 17, wherein the reductant is sodium ascorbate.

24. The kit of claim 17, wherein the structure-directing agent is polyethyleneimine.

25. The kit of claim 17, wherein the ratio of Cu ions to structure-directing agent in the copper growth reagent is 120:1 to 60:1.

26. The kit of claim 24, wherein the structure directing agent is polyethyleneimine (PEI), the copper ions are Cu²⁺, and the ratio of Cu²⁺ to PEI is 60:1.
